(19) United States
(12) Patent Application Publication
(10) Pub. No.: US 2020/0202103 A1
(43) Pub. Date: Jun. 25, 2020

(54) Method and Apparatus for Processing Retinal Images

(71) Applicant: University of Surrey, Guildford, Surrey (GB)

(72) Inventors: Hongying TANG, Guildford, Surrey (GB); Su WANG, Guildford, Surrey (GB)

(21) Appl. No.: 16/620,767

(22) PCT Filed: Jun. 8, 2018
(86) PCT No.: PCT/GB2018/051559

§ 371 Date: Dec. 9, 2019

(30) Foreign Application Priority Data
Jun. 9, 2017 (GB) 1709248.7

(51) Int. Cl. G06K 9/00; G06K 9/46; G06N 3/08; A61B 3/12

(52) U.S. Cl. CPC G06K 9/0061; G06K 9/00617; G06K 9/4619; G06K 2209/05; A61B 3/1233; A61B 3/1241; G06N 3/08

(57) ABSTRACT

Apparatus and methods for detecting features indicative of diabetic retinopathy in retinal images are disclosed. Image data of a retinal image is processed using a first convolutional neural network, to classify the retinal image as a normal image or a disease image, a feature of interest is selected from an image classified as a disease image, and image data of the selected feature is processed using a second convolutional neural network, to determine whether the selected feature is a feature indicative of diabetic retinopathy.

### TECHNICAL FIELD

The present invention relates to methods and apparatus for processing retinal images. More particularly, the present invention relates to detecting features indicative of diabetic retinopathy in retinal images.

### BACKGROUND

Diabetic retinopathy can be diagnosed by studying an image of the retina, and looking for types of lesion that are characteristic of diabetic retinopathy. Retinal images can be reviewed manually, however, the process is labour-intensive and is subject to human error. There has therefore been interest in developing automated methods of analysing retinal images in order to diagnose diabetic retinopathy.

The invention is made in this context.

### SUMMARY OF THE INVENTION

According to a first aspect of the present invention, there is provided apparatus for detecting features indicative of diabetic retinopathy in retinal images, the apparatus comprising a first convolutional neural network configured to process image data of a retinal image to classify the retinal image as a normal image or a disease image, a feature selection unit configured to select a feature of interest in an image classified as a disease image by the first convolutional neural network, and a second convolutional neural network configured to process image data of the selected feature to determine whether the selected feature is a feature indicative of diabetic retinopathy.

In some embodiments according to the first aspect, the feature selection unit is configured to crop the retinal image to obtain a lower-resolution cropped image which includes the selected feature of interest, and to pass the image data of the cropped image to the second convolutional neural network. For example, in one embodiment according to the first aspect, the feature selection unit is configured to select one of a plurality of predetermined image sizes according to a size of the feature of interest and to crop the retinal image to the selected image size, and the apparatus further comprises a plurality of second convolutional neural networks each configured to process image data for a different one of the plurality of predetermined image sizes, wherein the feature selection unit is configured to pass the image data of the cropped image to the corresponding second convolution neural network that is configured to process image data for the selected image size.

In some embodiments according to the first aspect, the feature selection unit is configured to determine the location of a feature of interest according to which nodes are activated in an output layer of the first convolutional neural network.

In some embodiments according to the first aspect, the first convolutional neural network is configured to classify the retinal image by assigning one of a plurality of grades to the retinal image, the plurality of grades comprising a grade indicative of a normal retina and a plurality of disease grades each indicative of a different diabetic retinopathy stage.

In some embodiments according to the first aspect, the plurality of disease grades comprises at least:
a first disease grade indicative of background retinopathy;
a second disease grade indicative of pre-proliferative retinopathy; and
a third disease grade indicative of proliferative retinopathy.

In some embodiments according to the first aspect, the second convolutional neural network is configured to classify the selected feature into one of a plurality of classes each indicative of a different type of feature that may be associated with diabetic retinopathy.

In some embodiments according to the first aspect, the plurality of classes comprises at least:
a first class indicative of a normal retina;
a second class indicative of a microaneurysm;
a third class indicative of a haemorrhage; and
a fourth class indicative of an exudate.

In some embodiments according to the first aspect, the feature selection unit is configured to apply a shade correction algorithm to identify one or more bright lesion candidates and/or dark lesion candidates in the retinal image as the selected feature of interest.

In some embodiments according to the first aspect, the first convolutional neural network comprises a plurality of first network layers comprising a plurality of convolutional layers and at least one max-pooling layer, a fully-connected layer connected to the last layer of the layer stack, and a softmax layer connected to the fully-connected layer, the softmax layer being configured to assign the retinal image to one of a plurality of predefined grades based on an output of the fully-connected layer.

In some embodiments according to the first aspect, the plurality of first network layers, the fully-connected layer and the softmax layer of the first convolutional neural network are configured as shown in FIG. 4.

In some embodiments according to the first aspect, the first convolutional neural network comprises a first plurality of layers comprising a plurality of convolutional layers and at least one max-pooling layer, a first fully-connected layer connected to the last layer of the first plurality of layers, and a first softmax layer connected to the first fully-connected layer, the first softmax layer being configured to assign the retinal image to one of a plurality of predefined grades based on an output of the first fully-connected layer.

In some embodiments according to the first aspect, the first plurality of layers, the first fully-connected layer and the first softmax layer of the first convolutional neural network are configured as shown in FIG. 4.

In some embodiments according to the first aspect, the second convolutional neural network comprises a second plurality of layers comprising a plurality of second convolutional layers and at least one second max-pooling layer, a second fully-connected layer connected to the last layer of the second plurality of layers, and a second softmax layer connected to the second fully-connected layer, the second softmax layer being configured to assign the retinal image to one of a plurality of predefined grades based on an output of the second fully-connected layer.

In some embodiments according to the first aspect, the second plurality of layers, the second fully-connected layer and the second softmax layer of the second convolutional neural network are configured as shown in FIG. 5.

In some embodiments according to the first aspect, the at least one first max-pooling layer and/or the at least one second max-pooling layer are configured to use a stride of 2.

In some embodiments according to the first aspect, the first convolutional neural network and/or the second convolutional neural network is configured to apply zero-padding after each convolutional layer.

According to a second aspect of the present invention, there is provided a method of detecting features indicative of diabetic retinopathy in retinal images, the method comprising steps of: processing image data of a retinal image using a first convolutional neural network, to classify the retinal image as a normal image or a disease image; selecting a feature of interest from an image classified as a disease image by the first convolutional neural network; and processing image data of the selected feature of interest using a second convolutional neural network, to determine whether the selected feature of interest is a feature indicative of diabetic retinopathy.

According to a third aspect of the present invention, there is provided a computer-readable storage medium arranged to store computer program instructions which, when executed, perform a method according to the second aspect.

### BRIEF DESCRIPTION OF THE DRAWINGS

Embodiments of the present invention will now be described, by way of example only, with reference to the accompanying drawings, in which:

FIG. 1 illustrates apparatus for detecting features indicative of diabetic retinopathy in retinal images, according to an embodiment of the present invention;

FIG. 2 is a flowchart showing a method of detecting features indicative of diabetic retinopathy in retinal images, according to an embodiment of the present invention;

FIG. 3 is a flowchart showing a method of selecting a feature of interest from a retinal image graded as a disease image, according to an embodiment of the present invention;

FIG. 4 illustrates the architecture of a first convolutional neural network for classifying a retinal image as a normal image or a disease image, according to an embodiment of the present invention; and

FIG. 5 illustrates the architecture of a second convolutional neural network for determining whether a feature of interest is a feature indicative of diabetic retinopathy, according to an embodiment of the present invention.

### DETAILED DESCRIPTION

In the following detailed description, only certain exemplary embodiments of the present invention have been shown and described, simply by way of illustration. As those skilled in the art would realize, the described embodiments may be modified in various different ways, all without departing from the scope of the present invention. Accordingly, the drawings and description are to be regarded as illustrative in nature and not restrictive. Like reference numerals designate like elements throughout the specification.

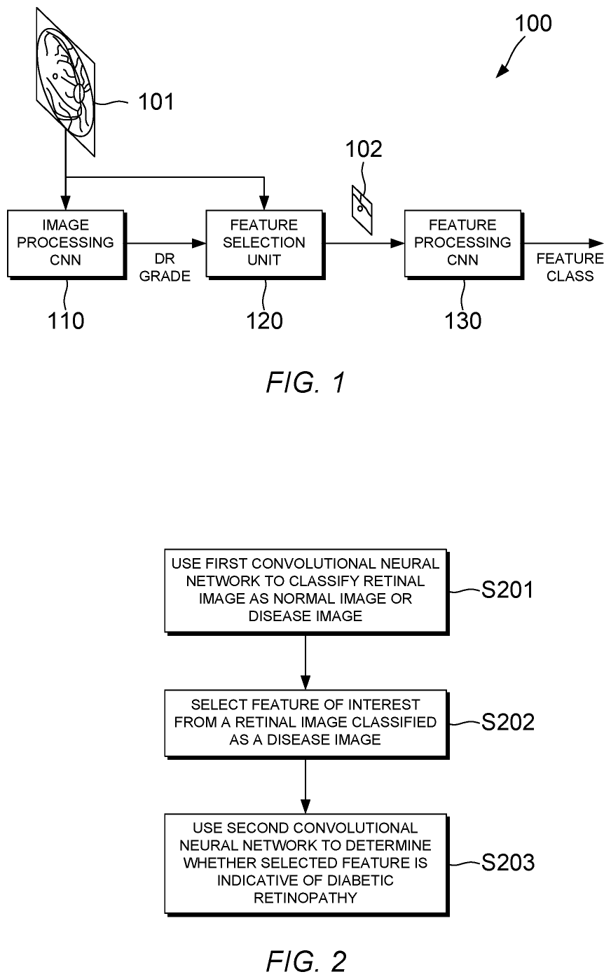

Referring now to FIGS. 1 and 2, an apparatus and method for detecting features indicative of diabetic retinopathy in retinal images are illustrated, according to an embodiment of the present invention. The apparatus 100 comprises a first convolutional neural network (CNN) 110, a feature selection unit 120, and a second CNN 130. Depending on the embodiment, the first CNN 110, the feature selection unit 120 and/or the second CNN 130 can be implemented in software or in hardware. When a software implementation is used, the apparatus 100 may comprise a processing unit comprising one or more processors, and memory in the form of a suitable computer-readable storage medium. The memory can be arranged to store computer program instructions which, when executed by the processing unit, perform the functions of one or more of the first CNN 110, the feature selection unit 120 and the second CNN 130.

As shown in FIG. 1, the apparatus 100 is provided with an input retinal image 101. In the present embodiment the apparatus is configured to receive an input image of size 512×512 pixels, but in other embodiments images of different sizes could be used. In step S201 of the flowchart shown in FIG. 2, the first CNN 110 processes image data of the input retinal image 101 in order to classify the retinal image 101 as a normal image or a disease image. The first CNN 110 may be referred to as an image processing CNN. Here, a ‘disease image’ refers to an image of a diseased retina, in particular, a retina of a subject suffering from diabetic retinopathy. In order to enable the first CNN 110 to distinguish between normal images and disease images, the first CNN 110 can be trained using a training data set which comprises images of healthy retinas and images of retinas at various stages of diabetic retinopathy. A method of training the first CNN 110 is described in more detail later with reference to FIG. 7.

The first CNN 110 processes the input retinal image 101 and outputs a grade assigned to the retinal image 101. The grade assigned to the image 101 indicates whether the image 101 has been classified as a normal image or as a disease image by the first CNN 110.

In the present embodiment, the first CNN 110 is configured to classify the retinal image 101 by assigning one of a plurality of grades to the retinal image, the plurality of grades comprising a grade indicative of a normal retina and a plurality of disease grades each indicative of a different diabetic retinopathy stage. In another embodiment, a single disease grade could be used instead of a plurality of grades indicative of different stages of disease, such that the image 101 is classified either as a normal image or as a disease image, without distinguishing between different disease stages.

The plurality of grades that may be assigned to a retinal image can be defined according to a medical classification scheme for grading the progression of diabetic retinopathy in a subject. Each grade may also be referred to as a class.

In the present embodiment, five grades are defined in accordance with the American Diabetic Retinopathy (DR) grading standard, as follows:
0=a normal grade, indicative of a normal retina;
1=a first disease grade, indicative of mild NPDR;
2=a second disease grade, indicative of moderate NPDR;
3=a third disease grade, indicative of severe NPDR; and
4=a fourth disease grade, indicative of proliferative retinopathy.
   where NPDR denotes non-proliferative diabetic retinopathy, which may also be referred to as background retinopathy, and PDR denotes proliferative diabetic retinopathy

In other embodiments, a different number of grades may be defined in accordance with a different classification scheme. For example, in another embodiment a total of four grades may be defined in accordance with the National Screening Committee (NSC) classification scheme used in England and Wales, as follows:
R0=a normal grade, indicative of a normal retina;
R1=a first disease grade, indicative of background retinopathy;
R2=a second disease grade, indicative of pre-proliferative retinopathy; and
R3=a third disease grade, indicative of proliferative retinopathy.

This classification scheme is similar to the American DR-based grading scheme described above, except that two grades are provided for background and pre-proliferative retinopathy as compared to the three grades provided for NPDR.

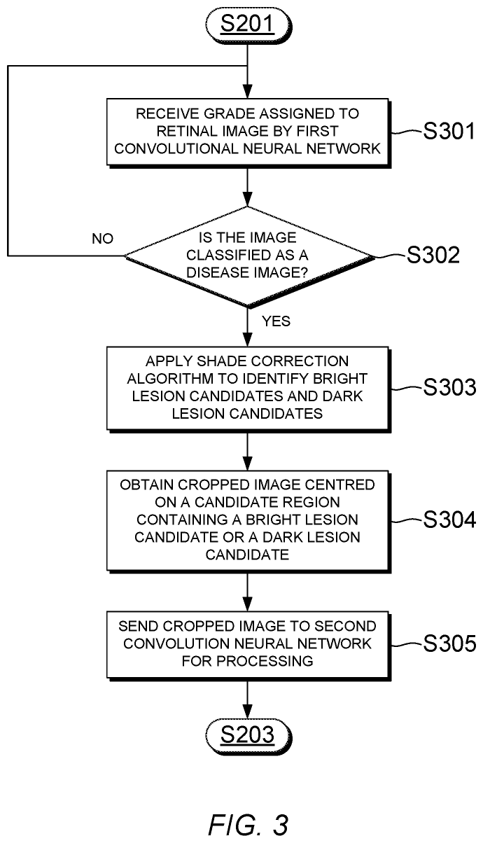

The feature selection unit 120 receives the grade outputted by the first CNN 110. Then, in step S202, in response to the grade indicating that the image 101 has been classified as a disease image, the feature selection unit 120 selects a feature of interest from the image 101. The feature of interest can be any candidate feature in the image 101 which might represent a lesion. In this context, a lesion refers to a region of the retina which has suffered damage as a result of diabetic retinopathy. The feature selection unit 120 can automatically select the feature of interest by identifying one or more candidate features in the image 101, and cropping the image 101 to obtain a smaller image 102 which contains one of the identified candidate features. In the present embodiment the image is cropped to a size of 61×61 pixels, but in other embodiments the cropped image 102 may have a different size. A method of selecting the feature of interest is described in more detail later with reference to FIG. 3.

Once a feature of interest has been selected, image data of the cropped image 102 which contains the feature of interest is sent to the second CNN 130 for further processing. The second CNN 130 may be referred to as a feature processing CNN. In step S203, the second CNN 130 processes image data of the cropped image 102 to determine whether the selected feature of interest is a feature that is indicative of diabetic retinopathy.

In the present embodiment, the second CNN 130 is configured to classify the cropped image 102 according to a type of feature that is present in the cropped image 102. The CNN 130 can be trained to recognise different types of features that may be associated with diabetic retinopathy. In the present embodiment the second CNN 130 is configured to assign one of a plurality of classes to the cropped image 102, as follows:
a non-lesion class indicative of a normal retina;
a first lesion class indicative of a microaneurysm (MA);
a second lesion class indicative of a haemorrhage; and
a third lesion class indicative of an exudate.

In other embodiments the second CNN 130 may be trained to detect different types of lesion, and/or to detect non-lesion features. For example, in another embodiment of the present invention the second CNN 130 may be configured to detect features such as drusen and/or vessels, instead of or in addition to the lesion features listed above. In some embodiments the second CNN 130 may be configured to just use a single lesion class, such that all types of lesion are grouped together in a single classification.

As described above, in some embodiments the second CNN 130 can be trained to detect non-lesion features such as drusen or blood vessels. Training the second CNN 130 to detect non-lesion features can reduce the risk of a false positive result. For example, drusen and exudates both appear as bright objects in retinal images. By training the second CNN 130 to distinguish between drusen and exudates, a situation can be avoided in which the second CNN 130 mixes drusen and exudates and returns a false positive result when only drusen are present. As a further example, in some cases abnormal new blood vessels (neovascularisation) can form at the back of the eye as part of proliferative diabetic retinopathy (PDR). The new blood vessels are fragile and so may burst and bleed (vitreous haemorrhage), resulting in blurred vision. In some embodiments, the second CNN 130 can be trained to identify these new blood vessels before they burst, so that suitable corrective action can be taken.

The apparatus and method described above with reference to FIGS. 1 and 2 can be used to automatically detect lesions in retinal images, and to assign a disease grading to the retinal image. The lesions and the disease grading can have diagnostic value, enabling an accurate diagnosis of diabetic retinopathy in the subject from which the retinal image was taken. For example, when an image has been classified as a disease image, the apparatus may be configured to output a version of the retinal image 101 with any detected lesions highlighted or otherwise indicated, for example by displaying a box centred on a detected lesion or an arrow pointing at the location of the detected lesion. The outputted image may be reviewed by a human operator to confirm the diagnosis of diabetic retinopathy.

Referring now to FIG. 3, a flowchart showing a method of selecting a feature of interest from a retinal image graded as a disease image is illustrated, according to an embodiment of the present invention. The method can be performed by the feature selection unit 120 of the apparatus 100 shown in FIG. 1, in order to identify and select features of interest from a disease image.

First, in step S301 the feature selection unit 120 receives the grade that has been assigned to the retinal image 101 by the first CNN 110. Then, in step S302 the feature selection unit 120 checks whether the assigned grade is a disease grade, that is, a grade that is indicative of a disease image. In the present embodiment, grades R1 to R3 are disease grades and grade R0 is a normal grade. If the image 101 has been graded as a normal image, then the process returns to the start and waits for the next image to be processed by the first CNN 110. On the other hand, if the image 101 has been graded as a disease image, then the process continues to step S303.

In step S303, the feature selection unit 120 applies a shade correction algorithm to identify bright lesion candidates and dark lesion candidates. In the present embodiment shade correction can be applied to individual colour channels in the input retinal image 101. For example, shade correction can be performed by applying a Gaussian filter to the image data for one colour channel of the retinal image 101, estimating a background image, and subtracting the background image from the Gaussian filtered image. This process results in a high-contrast image in which the background appears dark, and candidate lesions appear as features which are either much brighter or much darker than the background.

Although in the present embodiment a shade correction algorithm is used in step S303, in other embodiments a different method of identifying candidate lesions may be employed. For example, in another embodiment dark lesion candidates may be identified by searching for local minima in the retinal image, since dark lesions are darker than their surrounding background, and hence a local minima can be regarded as a candidate dark lesion. Conversely, bright lesion candidates may be identified by searching for local maxima in the retinal image. In another embodiment, a brute-force approach can be adopted by using all pixels in the retinal image as candidate lesions, so that every part of the retinal image will be analysed as if it contained a candidate lesion.

A feature with a high brightness relative to the background may be referred to as a bright lesion candidate, and a feature with a low brightness relative to the background may be referred to as a dark lesion candidate. Bright and dark lesion candidates constitute features of interest in the retinal image which may or may not represent lesions. The second CNN 130 can be used to analyse each feature of interest to determine whether or not it is a lesion.

In the present embodiment, once one or more candidate features have been identified in the retinal image 101, then in step S304 the feature selection unit 120 selects one of the candidate features to be processed by the second CNN 130 and obtains a lower-resolution cropped image 102 which includes the selected feature. The cropped image 102 may be centred on the selected feature. Then, in step S305 the image data of the cropped image 102 is sent to the second CNN 130 to be processed.

In some embodiments, the feature selection unit 120 can be configured to select one of a plurality of predetermined image sizes according to a size of the feature of interest, and to crop the retinal image to the selected image size. In a retinal image of size 512×512 pixels the features of interest may vary in diameter from about 5 pixels up to about 55 pixels. For example, an MA may have a size of about 5-10 pixels in a 512×512 input image, dot and blob haemorrhages may have sizes of around 5-55 pixels, exudates may have a size of around 5-55 pixels, and other features may have sizes of around 1-25 pixels. In one embodiment, the feature selection unit 120 may be configured to select from a plurality of predetermined image sizes including 15×15, 30×30, and 61×61 pixels. It will be appreciated that these resolutions are merely provided by way of an example, and other image sizes may be used for the cropped image in other embodiments.

Furthermore, in embodiments where the feature selection unit 120 can choose one of a plurality of predetermined image sizes for the cropped image, the apparatus can further comprise a plurality of second CNNs 130 each configured to process image data for a different one of the plurality of predetermined image sizes. The feature selection unit 120 can be configured to pass the image data of the cropped image to the corresponding second CNN 130 that is configured to process image data for the selected image size. In this way, the feature selection unit can automatically select the smallest possible one of the available image sizes that is suitable for the current feature of interest, and process the cropped image using a suitably-sized second CNN 130. A CNN which is configured to process a lower-resolution image can contain fewer layers, and fewer kernels within each layer, than a CNN which is configured to process a higher-resolution image. Accordingly, providing a plurality of second CNNs for different cropped image sizes can enable more efficient use of computing resources, by allowing the apparatus to choose a suitable image size for the current feature of interest.

By sending a cropped image 102 to the second CNN 130, the time taken to analyse the feature in the second CNN 130 can be decreased, since each layer in the second CNN 130 can include fewer kernels than if the full-resolution image was used. However, in other embodiments the second CNN 130 could be configured to process an image with the same resolution as the input retinal image 101, for example by centring the image on the selected feature and then padding the image borders with pixels having the same average brightness as the background.

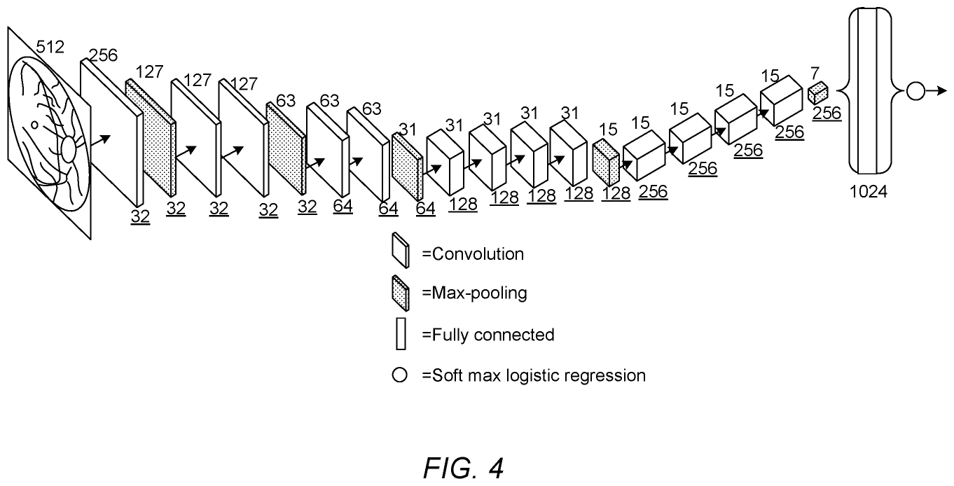

Referring now to FIG. 4, an example of the architecture of the first CNN 110 is illustrated, according to an embodiment of the present invention. In the present embodiment the first CNN 110 is configured to output one of five classes defined according to the American-DR grading standard described above, and is configured to receive an input image of size 512×512 pixels. However, in another embodiment the first CNN 110 may be configured to process a different size of image, and/or may be configured to use a different number of classes.

As shown in FIG. 4, the first CNN 110 of the present embodiment comprises a first plurality of layers comprising a total of thirteen convolutional layers and five max-pooling layers, a fully-connected layer connected to the last max-pooling layer, and a softmax layer connected to the fully-connected layer. The softmax layer is configured to assign the retinal image to one of the five grades defined based on the American DR grading standard, based on an output of the fully-connected layer. Details of the layer configuration of the first CNN 110 are shown below in Table 1:

TABLE 1

| Layer | Operation Name | Input Size | Parameters |
|---|---|---|---|
| 1 | convolution | 512 × 512 | F = 3, S = 2, K = 32 |
| 2 | max-pooling | 256 × 256 | F = 3, S = 2, K = 32 |
| 3 | convolution | 127 × 127 | F = 3, S = 1, K = 32 |
| 4 | convolution | 127 × 127 | F = 3, S = 1, K = 32 |
| 5 | max-pooling | 127 × 127 | F = 3, S = 2, K = 32 |
| 6 | convolution | 63 × 63 | F = 3, S = 1, K = 64 |
| 7 | convolution | 63 × 63 | F = 3, S = 1, K = 64 |
| 8 | max-pooling | 63 × 63 | F = 3, S = 2, K = 64 |
| 9 | convolution | 31 × 31 | F = 3, S = 1, K = 128 |
| 10 | convolution | 31 × 31 | F = 3, S = 1, K = 128 |
| 11 | convolution | 31 × 31 | F = 3, S = 1, K = 128 |
| 12 | convolution | 31 × 31 | F = 3, S = 1, K = 128 |
| 13 | max-pooling | 31 × 31 | F = 3, S = 2, K = 128 |
| 14 | convolution | 15 × 15 | F = 3, S = 1, K = 256 |
| 15 | convolution | 15 × 15 | F = 3, S = 1, K = 256 |
| 16 | convolution | 15 × 15 | F = 3, S = 1, K = 256 |
| 17 | convolution | 15 × 15 | F = 3, S = 1, K = 256 |
| 18 | max-pooling | 15 × 15 | F = 3, S = 2, K = 256 |
| 19 | fully connected | 7 × 7 | 1024 nodes |
| 20 | softmax | 1024 × 1 | 5 classes |

In Table 1, F denotes the size of the kernels in each layer, S is the stride, and K is the number of kernels, which may also be referred to as the depth. The stride is the distance between the kernel centres of neighbouring neurones in a kernel map. When the stride is 2, the kernels jump 2 pixels at a time.

In the present embodiment, the first convolutional neural network is configured to apply zero-padding after each convolutional layer in order to preserve spatial resolution after convolution. Zero padding can be used to control the spatial size of the output volumes, preserving the spatial size of the input volume so that the input and output width and height are the same. In the present embodiment zero-padding of 1 extra pixel is applied in the first convolution layer, and zero-padding of 2 extra pixels is applied in the other convolution layers.

In some embodiments of the present invention, a larger number of kernels may be represented by multiple smaller number of kernels. For example, a 5×5 kernel can be represented by employing two 3×3 kernels. Employing multiple small-sized kernel layers with non-linear rectifications can makes the CNN more discriminative, and requires less parameters to be optimised. Therefore in the present embodiment a relatively small kernel size of 3×3 is used. Also, in the present embodiment a kernel size of 3×3 and a stride of 2 is used for the max-pooling layers, resulting in the size of the feature map being halved after each group of convolutional layers.

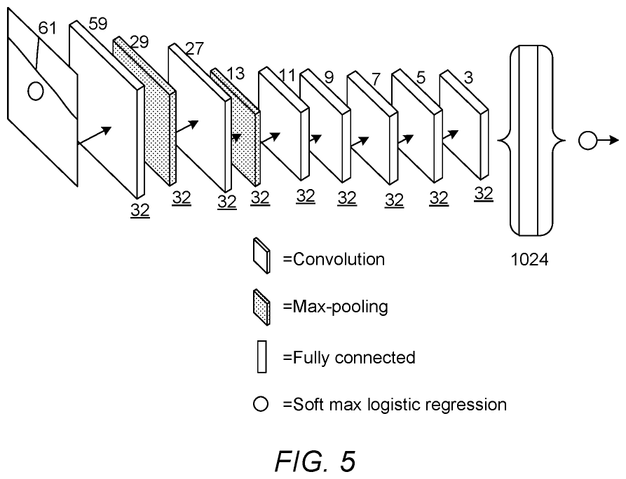

Referring now to FIG. 5, an example of the architecture of the second CNN 130 is illustrated, according to an embodiment of the present invention. In the present embodiment the second CNN 130 is configured to output one of four classes defined as described above, and is configured to receive an input image of size 61×61 pixels. However, in another embodiment the second CNN 130 may be configured to process a different size of image, and/or may be configured to use a different number of classes.

In some embodiments, the first CNN 1100 can be analysed backwards from the activated output class to the input image data, to determine the locations of features that cause the final activation in the output layer. The apparatus can then determine the location of a feature of interest according to which nodes in the output layer are activated.

For example, in a grade 1 image (only MAs present), only nodes that are associated with MAs will be activated. When certain nodes are activated, the location of the MA which triggered the activation of those nodes can be determined by analysing the structure of the first CNN 110 to determine which pixels are used as inputs for the activated nodes. This analysis can be performed during the training phase for the first CNN 110, to determine which nodes are activated when an MA is present in a particular region of the input image. The resulting information can be stored in a suitable format, for example in a look-up table (LUT), in which certain combinations of activated nodes are associated with a particular location of a candidate feature in the input image. This information may generally be referred to as feature location information. Then, when an unknown input image is processed and assigned a disease grade (grades 1 to 4 in the present embodiment), the feature selection unit 120 can check which nodes are activated, and compare the identified nodes to the stored feature location information to determine the location of a candidate feature in the input image. The feature selection unit 120 can then crop a corresponding part of the input image and send the cropped image to the second CNN 130 for further processing, as described above.

As shown in FIG. 5, the second CNN 130 of the present embodiment comprises a second plurality of layers comprising a total of seven convolutional layers and two max-pooling layers, a fully-connected layer connected to the last convolutional layer, and a softmax layer connected to the fully-connected layer. The softmax layer is configured to assign the retinal image to one of four classes indicating whether the feature is a non-lesion, a microaneurysm, a haemorrhage, or an exudate, based on an output of the fully-connected layer. Details of the layer configuration of the second CNN 130 are shown below in Table 2:

TABLE 2

| Layer | Operation Name | Input Size | Parameters |
|---|---|---|---|
| 1 | convolution | 61 × 61 | F = 3, S = 1, K = 32 |
| 2 | max-pooling | 59 × 59 | F = 3, S = 2, K = 32 |
| 3 | convolution | 29 × 29 | F = 3, S = 1, K = 32 |
| 4 | max-pooling | 27 × 27 | F = 3, S = 2, K = 32 |
| 5 | convolution | 13 × 13 | F = 3, S = 1, K = 32 |
| 6 | convolution | 11 × 11 | F = 3, S = 1, K = 32 |
| 7 | convolution | 9 × 9 | F = 3, S = 1, K = 32 |
| 8 | convolution | 7 × 7 | F = 3, S = 1, K = 32 |
| 9 | convolution | 5 × 5 | F = 3, S = 1, K = 32 |
| 10 | fully connected | 3 × 3 | 1024 nodes |
| 11 | softmax | 1024 × 1 | 4 classes |

As with the first CNN 110, in the present embodiment the second CNN 130 is configured to use a stride of 2 for each max-pooling layer. The second CNN 130 is also configured to use a small kernel size of 3×3 pixels for each of the convolutional and max-pooling layers, and is configured to apply zero-padding after each convolutional layer.

It should be understood that the architectures shown in FIGS. 4 and 5 are provided for illustrative purposes only, and should not be construed as being limiting. In other embodiments a different architecture may be used. For example, if a different input image size is used, the layer sizes, number of layers, and sequence of max-pooling and convolutional layers, may be modified accordingly.

In embodiments of the present invention, the first and second CNNs 110, 130 can be trained using suitable training data sets. The skilled person will be familiar with methods of training neural networks, and a detailed explanation will not be provided here. In the present embodiment, the first and second CNNs 110, 130 are trained using negative samples extracted only from images which do not include any lesions, and using positive samples extracted only from disease images at lesion locations (e.g. images including MAs, haemorrhages and/or exudates). The negative samples can be chosen so as to contain common interfering candidate objects, such as optic disc, vessel bifurcations and crossings, small disconnected vessels fragments and retinal haemorrhages, so as to train the CNN to distinguish features of interest from these interfering candidate objects.

The training images can be split into a training set and a validation set. For example, 90% of the training images can be allocated to the training set, and 10% can be allocated to the validation set. After training the CNN using the training set, the trained CNN can be used to analyse the validation set in order to confirm that the CNN has been trained correctly, by testing whether the expected results are obtained for the images in the validation set. Corresponding cropped training images centred on the lesion location, e.g. with image sizes of 61×61 pixels and three channels depth, can be generated in order to train the second CNN 130. In some embodiments, same data augmentation methods can be used to artificially increase the number of lesion samples available for training the second CNN 130. When training the first and second CNNs 110, 130, normal image patches can be randomly flipped horizontally and vertically to avoid possible over-fitting, which each resulting flipped image being given the same class label as the original patch.

Whilst certain embodiments of the invention have been described herein with reference to the drawings, it will be understood that many variations and modifications will be possible without departing from the scope of the invention as defined in the accompanying claims.

1. Apparatus for detecting features indicative of diabetic retinopathy in retinal images, the apparatus comprising:
a first convolutional neural network configured to process image data of a retinal image to classify the retinal image as a normal image or a disease image;
a feature selection unit configured to select a feature of interest in an image classified as a disease image by the first convolutional neural network; and
a second convolutional neural network configured to process image data of the selected feature to determine whether the selected feature is a feature indicative of diabetic retinopathy.

2. The apparatus of claim 1, wherein the feature selection unit is configured to crop the retinal image to obtain a lower-resolution cropped image which includes the selected feature of interest, and to pass the image data of the cropped image to the second convolutional neural network.

3. The apparatus of claim 2, wherein the feature selection unit is configured to select one of a plurality of predetermined image sizes according to a size of the feature of interest and to crop the retinal image to the selected image size, the apparatus further comprising:
a plurality of second convolutional neural networks each configured to process image data for a different one of the plurality of predetermined image sizes,
wherein the feature selection unit is configured to pass the image data of the cropped image to the corresponding second convolution neural network that is configured to process image data for the selected image size.

4. The apparatus of claim 1, wherein the feature selection unit is configured to determine the location of a feature of interest according to which nodes are activated in an output layer of the first convolutional neural network.

5. The apparatus of claim 1, wherein the first convolutional neural network is configured to classify the retinal image by assigning one of a plurality of grades to the retinal image, the plurality of grades comprising a grade indicative of a normal retina and a plurality of disease grades each indicative of a different diabetic retinopathy stage.

6. The apparatus of claim 5, wherein the plurality of disease grades comprises at least:
a first disease grade indicative of background retinopathy;
a second disease grade indicative of pre-proliferative retinopathy; and
a third disease grade indicative of proliferative retinopathy.

7. The apparatus of claim 1, wherein the second convolutional neural network is configured to classify the selected feature into one of a plurality of classes each indicative of a different type of feature that may be associated with diabetic retinopathy.

8. The apparatus of claim 7, wherein the plurality of classes comprises at least:
a first class indicative of a normal retina;
a second class indicative of a microaneurysm;
a third class indicative of a haemorrhage; and
a fourth class indicative of an exudate.

9. The apparatus of claim 1, wherein the feature selection unit is configured to apply a shade correction algorithm to identify one or more bright lesion candidates and/or dark lesion candidates in the retinal image as the selected feature of interest.

10. The apparatus of claim 1, wherein the first convolutional neural network comprises:
a first plurality of layers comprising a plurality of convolutional layers and at least one max-pooling layer;
a first fully-connected layer connected to the last layer of the first plurality of layers; and
a first softmax layer connected to the first fully-connected layer, the first softmax layer being configured to assign the retinal image to one of a plurality of predefined grades based on an output of the first fully-connected layer.

11. The apparatus of claim 10, wherein the first plurality of layers, the first fully connected layer and the first softmax layer of the first convolutional neural network are configured as shown in FIG. 4.

12. The apparatus of claim 1, wherein the second convolutional neural network comprises:
a second plurality of layers comprising a plurality of second convolutional layers and at least one second max-pooling layer;
a second fully-connected layer connected to the last layer of the second plurality of layers; and
a second softmax layer connected to the second fully-connected layer, the second softmax layer being configured to assign the retinal image to one of a plurality of predefined grades based on an output of the second fully-connected layer.

13. The apparatus of claim 12, wherein the second plurality of layers, the second fully-connected layer and the second softmax layer of the second convolutional neural network are configured as shown in FIG. 5.

14. The apparatus of claim 10, wherein the at least one first maxpooling layer and/or the at least one second max-pooling layer are configured to use a stride of 2.

15. The apparatus of claim 10, wherein the first convolutional neural network and/or the second convolutional neural network is configured to apply zero-padding after each convolutional layer.

16. A method of detecting features indicative of diabetic retinopathy in retinal images, the method comprising:
processing image data of a retinal image using a first convolutional neural network, to classify the retinal image as a normal image or a disease image;
selecting a feature of interest from an image classified as a disease image by the first convolutional neural network; and
processing image data of the selected feature of interest using a second convolutional neural network, to determine whether the selected feature of interest is a feature indicative of diabetic retinopathy.

17. A computer-readable storage medium arranged to store computer program instructions which, when executed, perform the method of claim 16.
